(19) United States
(12) Patent Application Publication
(10) Pub. No.: US 2025/0026885 A1
(43) Pub. Date: Jan. 23, 2025

(54) PERFLUOROPOLYETHER COPOLYMERS FOR BIOMEDICAL APPLICATIONS

(71) Applicant: Axoft, Inc., Cambridge, MA (US)

(72) Inventors: Foad Vashahi, Cambridge, MA (US); Andrew Robert Spencer, Cambridge, MA (US); Paul Claude Henri Le Floch, Cambridge, MA (US); Iraklis Kourtis, Arlington, MA (US)

(73) Assignee: Axoft, Inc., Cambridge, MA (US)

(21) Appl. No.: 18/778,677

(22) Filed: Jul. 19, 2024

Related U.S. Application Data
(60) Provisional application No. 63/514,755, filed on Jul. 20, 2023.

(51) Int. Cl. C08G 65/333; A61B 5/268; A61N 1/05; C08J 3/24

(52) U.S. Cl. CPC C08G 65/33355; A61B 5/268; C08J 3/24; A61B 2562/028; A61B 2562/04; A61N 1/05; C08J 2371/00

(57) ABSTRACT

Compositions and cross-linked polymer networks including at least one soft fluoropolymer segment and at least one hard fluorinated polymer segment which are covalently bonded through a linking moiety are provided. Methods of making the compositions and networks, and devices that incorporate them are also provided. The polymer networks may be designed to exhibit specific mechanical or physical properties which are tunable through by synthetic techniques including variation in the number and/or identity of the hard fluorinated polymer segments and soft fluoropolymer segments.

### INCORPORATION BY REFERENCE

An Application Data Sheet is filed concurrently with this specification as part of the present application. Each application that the present application claims benefit of or priority to as identified in the concurrently filed Application Data Sheet is incorporated by reference herein in their entireties and for all purposes.

### BACKGROUND

Perfluoropolyether (PFPE) materials, also referred to as perfluoropolymethyl-isopropyl-ethers, are synthesized as described in detail in W. C. Bunyard et al., Macromolecules 32, 8224 (1999). This process involves the creation of polymeric compounds composed of multiple, sequentially linked, fluorinated aliphatic ether units. The term “perfluoropolyether” refers to such polymers in which essentially all of the hydrogens have been replaced with fluorine. The fluorinated units can be linear (Z-type or D-type) or branched (K-type or Y-type) chains. The resulting PFPE materials are noted for their unique characteristics including being liquids at room temperature, having low surface energy and tunable clastic modulus, showing high gas permeability, high thermal stability, and high lubricity, and displaying low toxicity. They are resistant to chemical reactions and can be used in various environments as coatings, sealants, flexible fillers, and structural parts for a variety of medical applications.

Derivatization of these PFPE materials can be achieved through functionalizing with various groups such as epoxy, vinyl, hydroxyl, isocyanate, and amins. This can be subsequently cured via various well-known mechanisms including free and controlled radical, and epoxy ring opening polymerization methods. U.S. Pub. No. 2005/0142315 discloses PFPE material synthesis involving the methacrylate-functionalization of a commercially available PFPE diol with 2-isocyanato-ethyl methacrylate. The subsequent photocuring of the material is accomplished by blending it with 1 weight % of 2,2-dimethoxy-2-phenylacetophenone (DMPA) and exposing it to UV radiation. Moreover, U.S. Pub. No. 2005/0142315 discloses a novel use for liquid curable perfluoropolyether (PFPE) materials in medical applications, particularly where conventionally silicone has been used. The PFPE material is oxygen permeable and bacteria impermeable and may contain one or more pharmacological agents trapped within for delivery within the body of a subject. Some applications include being used in coatings, sealants, flexible fillers, and structural parts for a variety of medical applications.

The U.S. Pub. No. 2007/0178133 patent discloses the synthesis of PFPE and its derivatives. PFPE and its derivatives are synthesized by polymerizing perfluorinated monomers using catalysts such as cesium fluoride or photocatalysis by ultraviolet light, depending on the specific PFPE derivative being synthesized. Following their synthesis, these PFPE materials are end-capped with polymerizable groups for further functionalization. For instance, PFPE precursor can be end-capped with an acrylate, a methacrylate, an epoxy, an amino, a carboxylic, an anhydride, a maleimide, an isocyanato, an olefinic, or a styrenic group. The functionalized PFPE precursors are useful in forming a range of medical or surgical devices, with the chemical functionality introduced via the polymerizable group offering tunable properties such as hydrophobicity, reactivity, or the like. Furthermore, the patent also describes PFPE materials being used in a two-component liquid precursor system with other polymers like poly(dimethylsiloxane) (PDMS) for hybrid device fabrication. The patent discloses the synthesis of PFPE materials, their functionalization, and application in the formation of various medical devices. The utilization of these materials provides devices with unique properties such as low surface energy, high gas permeability, excellent release properties, solvent resistance, and biocompatibility.

U.S. Pat. No. 7,358,306 describes the synthesis of curable perfluoropolyether polyurethanes. The synthesis is initiated by reacting perfluoropolyether diols having a number average molecular weight ranging from 2,000 to 5,000. The perfluoropolyether diols have perfluorooxyalkylene units statistically distributed along the chain. These diols are then reacted with diisocyanates. The diisocyanates have the formula OCN—R—NCO, wherein R can be a hydrogen-containing and/or fluorinated radical. The diols and diisocyanates are further reacted with hydrogen-containing diols. These diols can be selected from one or more of the following: C₂-C₁₀ aliphatic diols, C₄-C₁₀ (alkyl)cycloaliphatic or (alkyl) aromatic diols, optionally comprising in the molecule two aliphatic or aromatic rings having six carbon atoms, and unsaturated aliphatic diols. Every reaction mentioned is conducted in the presence of a catalytic amount of an organometallic catalyst, in particular those of the organotin type. The resulting product is a perfluoropolyether polyurethane with improved flexibility at low temperatures, high chemical and solvent resistance, making them suitable for use in a variety of applications, including manufacturing medical devices and elastomers for various industries.

There are several underlining limitations with the prior art. First and foremost, materials for medical applications, need to be tunable over a wide range of properties to match, for example, the mechanical properties of different tissues. While prior disclosures have demonstrated PFPE materials' functionalization, the range and scope of their tunability are rather limited. Functions such as strength, elongation at break, adhesion, permeation, saturation, and electrical impedance, among others, all require formulations that can be tuned to match the unique needs of various biomedical applications.

Moreover, for biomedical applications, such as implantable electrodes, it is crucial that the materials closely resemble the properties of human tissues to ensure optimal compatibility and biointegration. Prior disclosures provide only a select range of properties that PFPE materials can emulate. PFPE materials and their derivatives, as disclosed before, lack the durability necessary to withstand the large, complex, and dynamic deformations that are commonplace inside the human body. Mechanical failure or functional degradation of a material inside the body or tear during surgical implantation can lead to critical complications.

Therefore, while the art has contributed significantly towards the advancement of PFPE materials, there remains a critical need for extending its tunability and durability, particularly in the context of biomedical applications.

The background description provided herein is for the purposes of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

The present invention relates generally to the field of material synthesis and, more specifically, to the synthetic processes and derivatization of PFPE materials for biomedical applications. This disclosure pertains to processes and systems for the creation of fluorinated polymers, which can include their use in different products such as but not limited to devices, sensors, implants, circuits, coated substrates, among others.

Methods for synthesizing PFPE crosslinked polymer networks and its derivatives with improved mechanical, chemical, and electrical properties are disclosed. These methods include chain extending PFPE oligomers with fluorinated diol or fluorinated diamine chain extenders, the first acting as the soft segment while the latter as the hard segment of the crosslinked polymer chain.

Compositions and cross-linked polymer networks including at least one soft fluoropolymer segment and at least one hard fluorinated polymer segment which are covalently bonded through a linking moiety are provided. The polymer networks have high tensile strength, improved toughness and increased elasticity as compared to non-segmented soft fluoropolymers. Methods of making the compositions and networks, and devices that incorporate them are also provided. The polymer networks may be designed to exhibit specific physical or mechanical properties which are tunable through by synthetic techniques including variation in the number and/or identity of the hard fluorinated and soft fluoropolymer segments.

Accordingly, in a first aspect, the present disclosure encompasses a composition. In some embodiments, the composition includes at least one soft fluoropolymer segment; and at least one hard fluorinated polymer segment; and where the at least one soft fluoropolymer segment and the at least one hard fluorinated polymer segment are covalently bound through a linking moiety having at least two reactive groups.

In some embodiments, the at least one soft fluoropolymer segment is poly(1,1,1,3,3,3,-hexafluoroisopropyl acrylate) (PHFIPA), poly[2-(perfluorohexyl)ethyl]acrylate, perfluoropolyether (PFPE), polytetrafluoroethylene (PTFE), tetrafluoroethylene propylene (TFE), perfluoropolyether dimethyl acrylate (PFPE-DMA), fluorinated ethylene-propylene (FEP), perfluoroalkoxy polymer (PFA), or polychlorotrifluoroethylene (PCTFE).

In some embodiments, the at least one soft fluoropolymer segment is a perfluoropolyether having a number average molecular weight Mn of from about 1,000 to about 10,000 g/mol.

In some embodiments, the perfluoropolyether also includes at least one cross-linkable moiety.

In some embodiments, the cross-linkable moiety is a methacrylate, an acrylate, or an epoxide.

In some embodiments, the linking moiety and the cross-linkable moiety are the same moiety.

In some embodiments, the linking moiety is a multi-functional isocyanate.

In some embodiments, the multi-functional isocyanate is isophorone diisocyanate (IPDI), hexamethylene diisocyanate (HDI), 4,4′-diisocyanato dicyclohexylmethane (HMDI), methylene diphenyl diisocyanate (MDI), 2,2′-MDI, 2,4′-MDI, 4,4′-MDI, toluene diisocyanate (TDI), 1,3,5-tris(6-isocyanatohexyl)-1,3,5-triazinanc-2,4,6-trione, 1,3,5-tris[(5-isocyanato-1,3,3-trimethylcyclohexyl)methyl]-1,3,5-triazinane-2,4,6-trione, 1,3,5-triazine-2,4,6 (1H,3H,5H)-trione or a combination thereof.

In some embodiments, the hard fluorinated polymer segment is formed of a fluorinated diol; and the fluorinated diol is hexafluoro-2,3-bis(trifluoromethyl)-2,3-butanediol, 2,2,3,3-tetrafluoro-1,4-butanediol (TFBD), 2,2,3,3,4,4-hexafluoro-1,5-pentanediol, 2,2,3,3,4,4,5,5-octafluoro-1,6-hexanediol, 2,2,3,3,4,4,5,5,6,6,7,7,8,8,9,9-hexadecafluoro-1,10-decanediol, 1H,1H,10H,10H-perfluoro-1,10-decanediol, or a combination thereof.

In some embodiments, the linking moiety is isophorone diisocyanate.

In a second aspect, the present disclosure encompasses a polymer network. In some embodiments, the polymer network includes soft fluoropolymer segments; and hard fluorinated polymer segments; and where soft fluoropolymer segments and hard fluorinated polymer segments are covalently bound through a linking moiety comprising at least two reactive groups; and where the soft fluoropolymer segments are connected through cross-linkers.

In some embodiments, the polymer network has a dielectric constant of from about 1 to about 5.

In some embodiments, the polymer network has a Young's modulus of from about 1 MPa to about 100 MPa.

In some embodiments, the polymer network has a dielectric constant of from about 1.5 to about 3 and a Young's modulus of from about 25 MPa to about 50 MPa.

In some embodiments, the polymer network is transparent to UV and visible light wavelengths.

In some embodiments, the polymer network has an optical refractive index of from about 1.2 to 1.4.

In some embodiments, the polymer network is an electrical insulator for a range of frequencies of from about 0.1 kHz to about 1 MHz.

In some embodiments, the polymer network has low permeability to water.

In some embodiments, the polymer network has a number average molecular weight of from about 1,100 to about 1,000,000 g/mol.

In some embodiments, the polymer network exhibits mechanical properties of high tensile strength, improved toughness, and increased elasticity.

In some embodiments, the polymer network has an elongation at break of from about 50% to about 150%.

In some embodiments, the polymer network exhibits strong adhesion to metal surfaces and thin films.

In some embodiments, the polymer network is stable at a temperature of up to 300° C.

In some embodiments, the polymer network can be used in electrodes, brain implants, coatings, or microelectromechanical systems (MEMS) devices.

In a second aspect, the present disclosure encompasses a device. In some embodiments, the device includes a polymer network and a microelectrode array.

In some embodiments, the polymer network encapsulates the microelectrode array to monitor or stimulate a tissue or an organ electrically.

In some embodiments, the organ or the tissue is brain, central nervous system, spinal cord, skeletal muscle, heart muscle, skin, liver, nasal cavity, spleen, diaphragm, lungs, thyroid, adrenal glands, stomach, eyes, thymus gland, lymph nodes, pancreas, small intestine, ureters, large intestine, bladder, gallbladder, lymphatic vessel, placenta, skeletal muscles, uterus, mouth, prostate, mesentery, pineal gland, subcutaneous tissue, colon, hypothalamus, mammary glands, pituitary gland, cervix, interstitium, parathyroid glands, tonsils, kidneys, or a combination thereof.

In a third aspect, the present disclosure encompasses a method of making a polymer network. In some embodiments, the method includes providing at least one soft fluoropolymer having reactive end groups; attaching a linking moiety through the reactive end groups of the at least one soft fluoropolymer to form a linkable soft fluoropolymer segment; polymerizing a fluorinated moiety to form a hard fluorinated polymer segment having reactive end groups; covalently bonding the linkable soft fluoropolymer segment to the hard fluorinated polymer segment having reactive end groups to form a hard- and soft-segmented fluorinated polymer composition; reacting the hard- and soft-segmented fluorinated polymer composition with a cross-linkable moiety to form a hard- and soft-segmented fluorinated polymer composition with cross-linkable end groups; and cross-linking the hard- and soft-segmented fluorinated polymer composition with cross-linkable end groups to form a polymer network.

In some embodiments, the at least one soft fluoropolymer is poly(1,1,1,3,3,3,-hexafluoroisopropyl acrylate) (PHFIPA), poly[2-(perfluorohexyl)ethyl]acrylate, perfluoropolyether (PFPE), polytetrafluoroethylene (PTFE), tetrafluoroethylene propylene (TFE), perfluoropolyether dimethyl acrylate (PFPE-DMA), fluorinated ethylene-propylene (FEP), perfluoroalkoxy polymer (PFA), or polychlorotrifluoroethylene (PCTFE).

In some embodiments, the at least one soft fluoropolymer is a K-type, a D-type, a Y-type, or a Z-type perfluoropolyether having a number average molecular weight Mn of from about 1,000 to about 10,000 g/mol.

In some embodiments, the cross-linkable moiety is an acrylation or methacrylation reagent; and wherein the hard- and soft-segmented fluorinated polymer composition with cross-linkable end groups is cross-linked into a polymer network by free radical polymerization in the presence of a thermal initiator, a photoinitiator, or a combination thereof.

In some embodiments, the cross-linkable moiety is an epoxy reagent; and where the hard- and soft-segmented fluorinated polymer composition with cross-linkable end groups is cross-linked into a polymer network using a photoacid generator.

In some embodiments, the fluorinated moiety is a fluorinated diamine, a fluorinated diisocyanate, a fluorinated diol or a combination thereof.

In some embodiments, each of the fluorinated moiety and the linking moiety are the same.

In some embodiments, each of the fluorinated moiety and the linking moiety are the same fluorinated or perfluorinated diisocyanate.

In some embodiments, covalent bonding of the linkable soft fluoropolymer segment to at least one hard fluorinated polymer segment is catalyzed by dibutyltin dilaurate (DBTDL).

In some embodiments, increasing the number of hard fluorinated polymer segments in the polymer network controls the mechanical properties of the polymer network.

In some embodiments, the method also includes sterilization of the polymer network by gamma irradiation, e-beam irradiation, ethylene oxide, chlorine dioxide, nitrogen dioxide, hydrogen peroxide, UV irradiation, dry heat, steam or a combination thereof.

In some embodiments, the method also includes patterning the polymer network on a substrate with a developing solvent, to provide a pattern with a lateral and vertical resolution below 100 μm.

In some embodiments, the method also includes processing the polymer network by injection molding, spin coating, dip coating, solvent casting, extrusion, electrospinning, thermal drawing, hot embossing, inkjet printing, stereolithography, fused deposition molding, imprinting or a combination thereof.

In some embodiments, the method also includes purifying the polymer network by precipitation.

These and other aspects are described further below with reference to the drawings.

### BRIEF DESCRIPTION OF THE DRAWINGS

FIG. 1A is a schematic representation of a polymeric composition in accordance with certain disclosed embodiments having soft fluorinated segments and hard fluorinated segments connect through a linking moiety; FIG. 1B is a schematic representation of the polymeric composition of FIG. 1A modified by functional groups which are appended to the ends of the hard fluorinated segments in accordance with certain disclosed embodiments; and FIG. 1C is a schematic representation of a polymer network formed from the polymeric composition of FIG. 1B after cross-linking in accordance with certain disclosed embodiments.

FIG. 2 is a flow diagram illustrating a method for forming a polymer network, in accordance with certain disclosed embodiments.

FIG. 3 is a reaction scheme illustrating the synthetic route of Example 1 for forming a polymer network, in accordance with certain disclosed embodiments.

FIG. 4 is a reaction scheme illustrating the synthetic route of Example 3 for forming a polymer network, in accordance with certain disclosed embodiments.

FIG. 5 illustrates average stress-strain plots of three crosslinked polymer network formulations; dotted line is a methacrylated Z-type crosslinked polymer network with average MW 10 kDa, dashed line is a methacrylated K-type crosslinked polymer network with average MW 10 kDa, and solid line a crosslinked polymer network synthesized according to Example 1 with average MW 10 kDa. The molecular weight for all polymers is calculated from their ¹⁹Fluorine NMR in accordance with certain disclosed embodiments.

FIG. 6 illustrates stress-strain plots of four crosslinked polymer network formulations: the solid line is a crosslinked polymer network prepared according to Example 1 (copolymer A) containing hard and soft fluorinated segments with molecular weight of 2.5 kDa, the dotted, dashed and dashed dotted lines are crosslinked polymer networks prepared according to Example 4 (copolymer B), Example 5 (copolymer C) and Example 6 (copolymer D), containing same soft fluorinated segments but different fluorinated diol with a lower, same and higher hard segment ratios than the crosslinked polymer network in Example 1, respectively in accordance with certain disclosed embodiments.

FIG. 7A is an optical micrograph showing a UV photopatterned thin film of a composition described herein after spin coating on a silicon oxide wafer and exposure through a photomask. The pattern “10” corresponds to lines of polymer of width 10 μm and spaced by 10 μm. The pattern “20” corresponds to lines of polymer of width 20 μm and spaced by 20 μm. The average thickness of the patterned layer is 1.6 μm when measured with a contact profiler in accordance with certain disclosed embodiments.

FIG. 7B is a scanning electron microscope (SEM) image of a 10 μm dense pattern from the top down in accordance with certain disclosed embodiments.

FIG. 7C is an SEM image of a 10 μm dense pattern cross section in accordance with certain disclosed embodiments.

FIG. 8A is a schematic of a microelectrode array encapsulated with a bottom and a top layer of a PFPE copolymer of the composition described herein. The schematic shows a side view of the device on a substrate of fabrication in accordance with certain disclosed embodiments.

FIG. 8B is a schematic of a microelectrode array encapsulated with a bottom and a top layer of a PFPE copolymer of the composition described herein. The schematic shows a top view of the device on a substrate of fabrication in accordance with certain disclosed embodiments.

FIG. 9 presents a UV absorbance plot of PFPE copolymer resin at 95 weight % concentration in a suitable solvent over wavelengths of 280 to 600 nm. The figure shows transparency and colorless properties of the resin within the visible region indicative of high efficiency of methacrylated polymer for UV curing in accordance with certain disclosed embodiments.

FIG. 10 provides data of a thermogravimetric analysis (TGA) of polymers described herein. TGA is a technique for the measurement of thermal stability of materials including polymers. It compares degradation of raw K-PFPE (solid line) and PFPE copolymer (dashed line) prepared according to Example 6 without performing the methacrylation step. For the same mass loss, PFPE copolymer shifts the thermal decomposition temperature and improves thermal stability by nearly 90 degrees Celsius.

FIG. 11 presents a differential scanning calorimetric (DSC) curve measuring how physical properties of a sample change, along with temperature. It compares performance of methacrylated K-PFPE (dashed line) against PFPE copolymer resin prepared according to Example 6 without performing the crosslinking step. The data indicates glass transition temperatures well below room temperature for both materials, indicating PFPE copolymer is a liquid at room temperature and yields elastomeric properties upon crosslinking.

FIG. 12 presents images of crosslinked polymer networks prepared in different methods in accordance with certain disclosed embodiments. On the left side of the image, a transparent cube prepared through crosslinking of the resin in a cube mold. On the right, a complex geometrical object resembling a brain prepared through crosslinking of the resin in a brain mold. Resin penetrates in narrow pathways due to its liquid nature and allows preparing complex objects. On the bottom is an image of a dogbone shaped object prepared through crosslinking of polymer in a rectangular mold followed by extraction of the dogbone via die cutter.

FIG. 13 is plot of dielectric constant measurement of PFPE copolymer 1 and 2 compared with K-PFPE and Z-PFPE samples measured using a two parallel plate method. The measurement was done over the range of 100 Hz to 1 MHz frequencies at which PFPE copolymer shows range of dielectric constant between 2.5 and 1.9, respectively.

FIG. 14 compares stress-strain plots of six crosslinked polymer network formulations before and after sterilization using chlorine dioxide: the crosslinked polymer network prepared according to Example 1. Pre-# lines illustrate mechanical properties of the measured dogbones before sterilization and post-# curves show mechanical properties of 3 different dogbones cut from the same elastomer after sterilization. Stiffness of elastomer before and after sterilization remains unchanged.

### DETAILED DESCRIPTION

In the following description, numerous specific details are set forth to provide a thorough understanding of the presented embodiments. The disclosed embodiments may be practiced without some or all of these specific details. In other instances, well-known process operations have not been described in detail to not unnecessarily obscure the disclosed embodiments. While the disclosed embodiments will be described in conjunction with the specific embodiments, it will be understood that it is not intended to limit the disclosed embodiments.

Large scale, brain-wide neuron activity mapping is important for deciphering neuronal population dynamics for neuroscience, understanding and alleviating neurological disorders, measuring neurological activity, providing neurological stimulation, building high-bandwidth brain machine interfaces (BMIs), for neuroprosthetics, and for communications. Ultimately, brain mapping aims to simultaneously record activities from millions, if not billions, of neurons at single-cell, millisecond spatiotemporal resolution in a chronically stable manner. “Tissue-like” thin-film electronics with subcellular feature size and tissue-level flexibility can provide for gliosis-free implantation, permit continuous stable recording of neuron activity at single-cell, single-spike spatiotemporal resolution for applications in neuroscience, bioelectronic medicine, brain-machine interfaces (BMIs), and the like.

One major challenge has been scaling up the number and spatial density of microelectrodes in tissue-like electronics without using rigid materials that are fundamentally mis-matched with the mechanical properties of the tissue into which they are implanted (e.g., brain tissue). Another challenge is the tendency of soft electronics to degrade in the brain's chemical environment, which can degrade many polymeric materials over time.

It was discovered that articles and sensors comprising fluorinated polymers, such as fluorinated elastomers including perfluorinated elastomers can offer a significant advantage for electronic devices such as neural implants. For example, fluorinated elastomers or other polymers may have desirable electrical and/or mechanical properties for implantation into soft tissues such as brain or other neural tissue and may exhibit exceptional long-term stability under physiological conditions.

Accordingly, the present disclosure recognizes the importance of fluorinated elastomers and other polymers for brain and other tissue implants, and provides inventive methods of preparing multilayered articles comprising, inter alia, multiple fluorinated elastomer or polymer layers for implants and other applications. These articles may demonstrate some of the outstanding properties provided by the utilization of fluorinated elastomers or other polymers. For example, some illustrative, non-limiting articles described herein comprise 0.05 electrodes per micron² and/or have an overall elastic modulus of less than or equal to 10⁶ Pa. It is believed that this number of electrodes per micron² represents a tenfold increase in the area number density of electrodes, relative to sensors with a comparable elastic modulus. Moreover, it is believed this elastic modulus represents a thousandfold reduction in the clastic modulus of a brain-sensor having a comparable number of electrodes per micron².

Although nanofabrication techniques can be used to produce bioelectronics for in vivo use, the long-term stability of these devices under physiological conditions, as well as the mismatch between their mechanical properties and the mechanical properties of human tissue, limit the scope of these technologies. In some embodiments, fluorinated polymers including fluorinated elastomers (such as perfluorinated elastomers) have been identified as a way to address these limitations. Thus, the present disclosure, in certain aspects, generally relates to fluorinated polymers such as fluorinated elastomers, and in certain embodiments, perfluorinated elastomers with long-term stability in near-physiological conditions that can be used in a variety of articles and devices. For example, in some embodiments, these fluorinated elastomers or other polymers are used for surgical implants, e.g., as coatings.

Some aspects of the present disclosure are directed to systems and methods of preparing fluorinated elastomers or other fluorinated polymers, including articles comprising such polymers, e.g., devices, sensors, implants, circuits, coated substrates, or the like. In addition, some aspects of the present disclosure are directed to systems and methods of preparing fluorinated polymers such as fluorinated elastomers (e.g., perfluorinated elastomers), including articles containing such polymers, e.g., devices, sensors, implants, circuits, coated substrates, or the like. Without being bound by any theory, it is believed that the super-hydrophobicity of fluorinated elastomers or other fluorinated polymers, and especially perfluorinated elastomers, can make fabrication of articles and devices comprising such polymers challenging. Thus, in certain embodiments, the present disclosure is directed towards methods of treating fluorinated polymers such as a fluorinated elastomer (e.g., a perfluoropolyether) that unexpectedly allows the deposition and stable bonding of additional material to the fluorinated elastomer or other polymer. In certain embodiments the fluorinated polymer such as fluorinated elastomer (e.g., perfluorinated elastomer) may be treated by applying a plasma (e.g., argon plasma, nitrogen plasma, oxygen plasma, CF₄ plasma, C₄F₈ plasma etc.) to the polymer. The additional material, in some cases, is additional fluorinated elastomer (or other polymer) that can increase the overall thickness of a perfluorinated layer. Thus, in some embodiments, the fabrication of surprisingly thick perfluorinated elastomer (or other polymer) layers (e.g., thicker than 300 nanometers) is disclosed. This surprising thickness may beneficially improve the stability and/or mechanical properties of fluorinated polymers such as perfluorinated elastomers in electronics. In contrast, other techniques are not able to produce such thick layers on articles or devices.

In certain embodiments the additional material(s) can comprise a material(s) other than the fluorinated elastomer or other fluorinated polymer. Such materials can include, but are not limited to, conductive materials. Illustrative conductive materials include, but are not limited to metals, metal alloys, metal oxides, metal nitrides, and the like (e.g., a metal selected from the group consisting of gold, platinum, iridium, tungsten, tantalum, tin, nichrome, titanium, copper, rhodium, rhenium, silver, stainless steel, palladium, aluminum, zirconium, conducting oxides or nitrides thereof, and alloys thereof, titanium nitride, platinum-iridium alloy, and the like), conductive polymers (e.g., polyacetylene, polypyrrole, polyindole, polyaniline and their copolymers), graphene), or conductive hydrogels (e.g. poly(2-acrylamido-2-methyl-1-propanesulfonic acid), poly(acrylic acid), poly[2-(Methacryloyloxy)ethyl]dimethyl-(3-sulfopropyl)ammonium hydroxide, and the like). In certain embodiments the additional material(s) can comprise semiconductor materials including, but not limited to group IV elemental semiconductors, (C, Si, Ge, Sn), group IV compound semiconductors, Group VI elemental semiconductors, (S, Se, Te), III-V semiconductors, II-VI semiconductors, I-VII semiconductors, IV-VI semiconductors, V-VI semiconductors, II-V semiconductors, I-III-VI2 semiconductors, semiconductor oxides. organic semiconductors, and the like, as well as other materials.

In certain aspects, the present disclosure is directed towards articles comprising fluorinated elastomers or other fluorinated polymers. Various non-limiting examples are provided herein for the purpose of illustration however, other embodiments are possible, and using the description provided herein will be available to one of skill in the art.

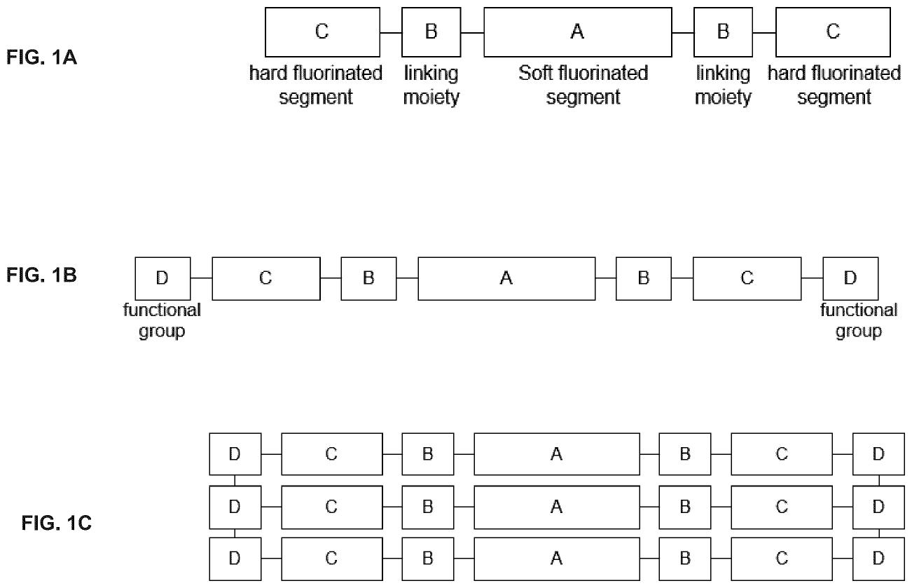

FIGS. 1A-1C provide an overview of the structures of polymer networks of the present disclosure, indicating how the various building blocks are connected. FIG. 1A is a schematic representation of a polymeric composition in accordance with certain disclosed embodiments having soft fluorinated polymer segments A and hard fluorinated polymer segments C connect through a linking moiety B; FIG. 1B is a schematic representation of the polymeric composition of FIG. 1A modified by functional groups D which are appended to the ends of the hard fluorinated polymer segments in accordance with certain disclosed embodiments; and FIG. 1C is a schematic representation of a polymer network formed from the polymeric composition of FIG. 1B after cross-linking of functional groups D in accordance with certain disclosed embodiments.

The schematic overview provides one configuration for the polymer networks. However, those skilled in the art understand that the components may be assembled in different configurations. For example, instead of the FIG. 1B configuration of D-C-B-A-B-C-D, configurations having two or more linked A groups which may be the same or different fluoropolymers (i.e. D-C-B-A¹-A²-B-C-D); or two or more linked B groups which may be the same or different hard fluorinated segments (i.e. D-C¹-C²-B-A-B-C²-C¹-D), or networks having a hard fluorinated polymer segment or segments between a soft fluoropolymer segment or segments (i.e D-A-B-C-B-A-D) may also be advantageous for certain applications.

The crosslinked polymer networks created typically exhibit enhanced yield strength and elongation at break, and improved dielectric properties over crosslinked polymer networks that are synthesized from similar branched (K-type or Y-type) or linear (Z-type or D-type) without the fluorinated chain extender (hard segments).

In some embodiments, the disclosure encompasses a perfluoropolyether copolymer composition having soft fluorinated segments and hard fluorinated segments which are covalently linked together. The composition may have a first component (A, soft fluorinated segment) which is a perfluoropolyether chain with an average molecular weight Mn between 1,000-10,000 g/mol with a functionality greater than one, and second component (B) which is a linker with a functionality greater than one, and a third component (C, hard fluorinated segment) which is a fluorinated segment with an average molecular weight Mn=100-10,000 g/mol with a functionality greater than one, or a repetition of (A), (B) and (C) segments. In some embodiments, the copolymer composition may be synthesized by end-capping component (A) with component (B), followed by linking the product with component (C). In some embodiments, component (A) is capped with component (B), which may be a multifunctional isocyanate such as isophorone diisocyanate (IPDI). In some embodiments, the final polymer chain length is controlled by the stoichiometry of the components (A), (B) and (C). In some embodiments, increasing the number of repeats of component (C) controls the mechanical properties of the polymer.

The PFPE copolymer may be capped with an acrylation or methacrylation reagent and crosslinked into a polymer network using free radical polymerization using thermal initiators or photoinitiators or a combination thereof; or capped with an epoxy reagent and crosslinked into a polymer network using a photoacid generator. In some embodiments, the component (A) is a K-type, a D-type, a Y-type, or a Z-type PFPE end-capped with IPDI as component (B) and chain extended with TFBD as component (C). In some embodiments, the components (B) and (C) are combined as a fluorinated or perfluorinated diisocyanate such as tetrafluoro-1,3-phenylene diisocyanate.

The crosslinked polymer network may be a crosslinked polymer network exhibiting enhanced yield strength, elongation at break, thermal stability, and improved dielectric properties compared to a crosslinked polymer network composed only of components (A) crosslinked by acrylating or methacrylating reagents. In some embodiments, the polymer network can withstand heat between 200° C. and 300° C. depending on the composition before any degradation or weight loss is observed.

In some embodiments, the method for making the polymer networks illustrated by FIG. 1C include chain extending PFPE oligomers with fluorinated diol or fluorinated diamine chain extenders, the first acting as the soft segment while the latter as the hard segment of the crosslinked polymer chain. In some embodiments, the fluorinated diol/diamine linker/chain extender can be fluorinated or perfluorinated diols such as but not limited to 2,3,5,6-tetrafluoro-4-(hydroxymethyl)phenyl]methanol, 2,2-bis(4-aminophenyl)hexafluoropropane, 2,2-bis(4-hydroxyphenyl)hexafluoropropane, hexafluoro-2,3-bis(trifluoromethyl)-2,3-butanediol, 2,2,3,3-tetrafluoro-1,4-butanediol (TFBD), 2,2,3,3,4,4-hexafluoro-1,5-pentanediol, 2,2,3,3,4,4,5,5-octafluoro-1,6-hexanediol, 2,2,3,3,4,4,5,5,6,6,7,7,8,8,9,9-hexadecafluoro-1,10-decanediol, 1H,1H,11H,11H-perfluoro-3,6,9-trioxaundecane-1,11-diol or 1H,1H,10H,10H-perfluoro-1,10-decanediol. In some embodiments of the disclosure, the PFPE can be a linear (Z-type or D-type) or branched (K-type or Y-type) perfluoropolyether and the molecular weight can vary from 1,000 Da to 10,000 Da. In some other embodiments of the disclosure, the PFPE is capped with aliphatic diisocyanates such as but not limited to isophorone diisocyanate (IPDI), hexamethylene diisocyanate (HDI), 4,4′-diisocyanato dicyclohexylmethane (HMDI) or aromatic diisocyanates such as but not limited to methylene diphenyl diisocyanate (MDI), 2,2′-MDI, 2,4′-MDI, 4,4′-MDI, toluene diisocyanate (TDI), 1,3,5-tris(6-isocyanatohexyl)-1,3,5-triazinane-2,4,6-trione, and 1,3,5-tris[(5-isocyanato-1,3,3-trimethylcyclohexyl)methyl]-1,3,5-triazinane-2,4,6-trione and 1,3,5-triazine-2,4,6 (1H,3H,5H)-trione. In one aspect of the disclosure, the final polymer length can be controlled by the stoichiometry of the PFPE, chain extender and diisocyanate, respectively. The molecular weight of such polymers can be adjusted from 1,000 Da to 100,000 Da.

In one embodiment, the chain extended PFPE can be capped with a reagent capable of radical polymerization such as but not limited to allyl isocyanate, 2-isocyanatoethyl methacrylate (IEM), 2-isocyanatoethyl acrylate, 3-Isopropenyl-α,α-dimethylbenzyl isocyanate. The polymerization of such prepolymers can be initiated using free radical polymerization using thermal initiators such as benzoyl peroxide, 2,2′-azobisisobutyronitrile (AIBN), 4,4′-Azobis(4-cyanopentanoic acid) (ACPA), 4,4′-azobis(4-cyanovaleric acid) (ACVA) or photoinitiators such as phenylbis(2,4,6-trimethylbenzoyl)phosphine oxide (BAPO), diphenyl(2,4,6-trimethylbenzoyl)phosphine oxide (TPO), ethyl(2,4,6-Trimethylbenzoyl)-phenyl phosphinate (TPO-L), lithium phenyl(2,4,6-trimethylbenzoyl) phosphinate (LAP), 2-hydroxy-4′-(2-hydroxyethoxy)-2-methylpropiophenone, 2-hydroxy-2-methylpropiophenone or any combination thereof.

In one embodiment, the chain extended PFPE can be capped with cyclic monomers amenable to ring opening polymerization including but not limited to epoxides, lactones, oxazolines, and lactams. The polymerization of such prepolymers can be initiated using anionic or cationic imitators such as nucleophilic reagents such as hydroxyl groups for anionic ring opening and photoacid generators and photobase generators including but not limited to triphenyl sulfonium hexafluoroantimonate, triphenyl sulfonium hexafluorophosphate, 2-(3,4-dimethoxystyryl)-4,6-bis(trichloromethyl)-1,3,5-triazine, or p-toluene sulfonic acid for cationic ring opening, respectively.

In one embodiment, the chain extended PFPE capped with reagents capable of radical polymerization such as but not limited to allyl isocyanate, 2-isocyanatoethyl methacrylate (IEM), 2-isocyanatocthyl acrylate, 3-isopropenyl-α,α-dimethylbenzyl isocyanate can be formulated by di-thiol or tetra-thiol moieties including but not limited to 1,6-hexanedithiol, 2,2′-(ethylenedioxy)diethanethiol, or pentaerythritol tetrakis(3-mercaptopropionate). The polymerization of such prepolymers can be initiated using free radical polymerization using thermal initiators such as benzoyl peroxide, 2,2′-azobisisobutyronitrile (AIBN), 4,4′-azobis(4-cyanopentanoic acid) (ACPA), 4,4′-azobis(4-cyanovaleric acid) (ACVA) or photoinitiators such as phenylbis(2,4,6-trimethylbenzoyl)phosphine oxide (BAPO), diphenyl(2,4,6-trimethylbenzoyl)phosphine oxide (TPO), ethyl(2,4,6-Trimethylbenzoyl)-phenyl phosphinate (TPO-L), lithium phenyl(2,4,6-trimethylbenzoyl) phosphinate (LAP), 2-hydroxy-4′-(2-hydroxyethoxy)-2-methylpropiophenone (Irgacure 2959), 2-hydroxy-2-methylpropiophenone (Irgacure 1173) or any combination thereof.

In one embodiment, the chain extended PFPE capped with reagents capable of radical polymerization such as but not limited to allyl isocyanate, 2-isocyanatoethyl methacrylate (IEM), 2-isocyanatocthyl acrylate, or 3-isopropenyl-α,α-dimethylbenzyl isocyanate can be formulated by addition of di, tri, and tetra functional crosslinkers including but not limited to 1,6-hexanediol diacrylate, polyethylene glycol dimethacrylate, pentaerythritol tetraacrylate. The polymerization of such prepolymers can be initiated using free radical polymerization using thermal initiators such as benzoyl peroxide, 2,2′-azobisisobutyronitrile (AIBN), 4,4′-azobis(4-cyanopentanoic acid) (ACPA), 4,4′-azobis(4-cyanovaleric acid) (ACVA) or photoinitiators such as phenylbis(2,4,6-trimethylbenzoyl)phosphine oxide (BAPO), diphenyl(2,4,6-trimethylbenzoyl)phosphine oxide (TPO), ethyl(2,4,6-trimethylbenzoyl)-phenyl phosphinate (TPO-L), lithium phenyl(2,4,6-trimethylbenzoyl) phosphinate (LAP), 2-hydroxy-4′-(2-hydroxyethoxy)-2-methylpropiophenone (Irgacure 2959), 2-hydroxy-2-methylpropiophenone (Irgacure 1173) or any combination thereof.

According to some embodiments of the present invention the PFPE is K-type (Rx) PFPE end-capped with IPDI and chain extended with TFBD. In one aspect of the invention the equivalents of TFBD per mole of PFPE are 1 and the equivalents of the IEM per mole of the previously synthesized intermediate product are 2.

In some other embodiment of the disclosure, the reaction is catalyzed using a catalyst such as but not limited to dibutyltin dilaurate (DBTDL), dibutyltin diacetate (DBTDA), and methanesulfonic acid. In the embodiments that the polymer chain is terminated with an acrylation reagent the same catalyst can be used throughout the reaction.

In one aspect of this disclosure, the diisocyanate capping and chain extension are combined in one step using a fluorinated or perfluorinated diisocyanate such as but not limited to tetrafluoro-1,3-phenylene diisocyanate.

In some embodiments, the raw polymer mix can be purified by first precipitating the polymer from its solvent using a non-solvent. The precipitated polymer is then recovered by centrifugation followed by rotary evaporator to remove solvent.

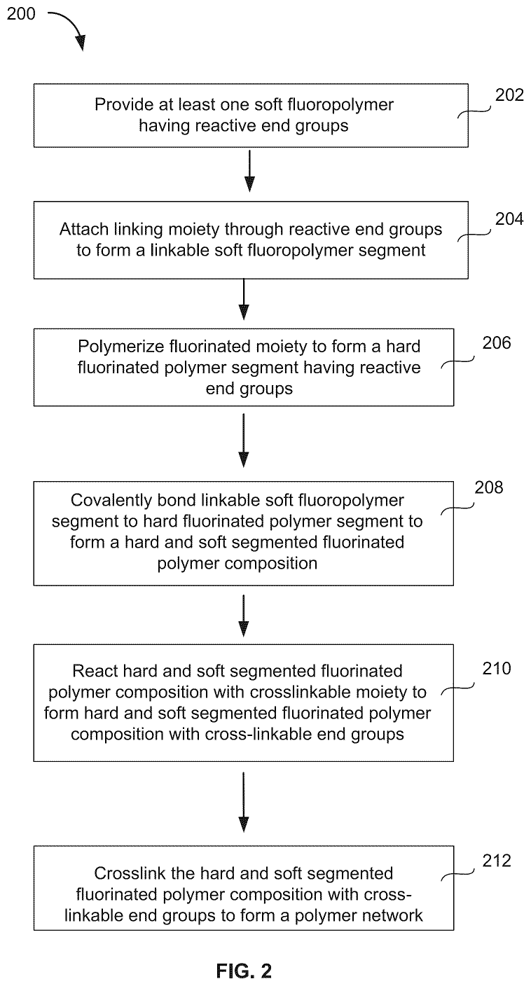

A further description of the preparation of the polymer networks is provided in conjunction with FIG. 2 below and in the Examples.

FIG. 2 is a flow diagram illustrating a method 200 for forming a polymer network, in accordance with certain disclosed embodiments. In operation 202, at least one soft fluoropolymer having reactive end groups is provided. Reactive end groups include, but are not limited to alcohols, amines, azides, thiols or isocyanate groups. Soft fluoropolymers include fluoropolymers which, when cross-linked into an elastomer, have an elastic modulus below 500 MPa.

In some embodiments, the soft fluoropolymer segment is an elastomer. Elastomers are polymers that are characterized by weak intermolecular forces and consequently viscoelasticity.

For instance, in some embodiments, the elastomer may exhibit a low elastic modulus. For example, in certain embodiments, the elastomer has an elastic modulus below 10 MPa, below 5 MPa, below 2 MPa, below 1 MPa, or lower. In some embodiments, the elastomer can exhibit a high elastic tensile deformation. For example, in some embodiments, the elastomer can exhibit clastic tensile deformation at or above 20% strain, 30% strain, 50% strain, or 100% strain. In some embodiments, combinations of these mechanical properties are possible. For example, in some embodiments, the elastomer has an elastic modulus below 1 MPa and can exhibit elastic tensile deformation at or above 20% strain. The clastic modulus and/or the elastic tensile deformation may be determined by any suitable method. For example, the elastic modulus and the elastic tensile deformation could be measured using a tensile tester.

The composition may contain one or more soft fluoropolymer segments, and when there is more than one soft fluoropolymer segment, the segments may be the same fluoropolymers or different fluoropolymers.

The soft fluoropolymer segment may be a K-type, a D-type, a Y-type, or a Z-type perfluoropolyether having a number average molecular weight Mn of from about 1,000 to about 10,000 g/mol. Suitable soft fluoropolymers are described in U.S. Patent Pub. Nos. 2024/0041376 and 2024/0131828, the disclosures of which are hereby incorporated by reference in their entireties.

In operation 204, a linking moiety is attached through the reactive group, such as through the oxygen of an alcohol end group, to form a linkable soft fluoropolymer segment. In some embodiments, the linking moiety is a multi-functional isocyanate such as a di-isocyanate. Suitable linking moieties include, but are not limited to, isophorone diisocyanate (IPDI), hexamethylene diisocyanate (HDI), 4,4′-diisocyanato dicyclohexylmethane (HMDI), methylene diphenyl diisocyanate (MDI), 2,2′-MDI, 2,4′-MDI, 4,4′-MDI, toluene diisocyanate (TDI), 1,3,5-tris(6-isocyanatohexyl)-1,3,5-triazinane-2,4,6-trione, 1,3,5-tris[(5-isocyanato-1,3,3-trimethylcyclohexyl)methyl]-1,3,5-triazinane-2,4,6-trione, 1,3,5-triazine-2,4,6 (1H,3H,5H)-trione, or a combination thereof.

In operation 206, the hard fluorinated polymer segment is formed by polymerization of a fluorinated moiety. In some embodiments, the fluorinated moiety is a fluorinated diamine, a fluorinated diisocyanate, a fluorinated diol or a combination thereof. Suitable fluorinated moieties include, but are not limited to, fluorinated diols such as hexafluoro-2,3-bis(trifluoromethyl)-2,3-butanediol, 2,2,3,3-tetrafluoro-1,4-butanediol (TFBD), 2,2,3,3,4,4-hexafluoro-1,5-pentanediol, 2,2,3,3,4,4,5,5-octafluoro-1,6-hexanediol, or 2,2,3,3,4,4,5,5,6,6,7,7,8,8,9,9-hexadecafluoro-1,10-decanediol, 1H,1H,10H,10H-perfluoro-1,10-decanediol.

The hard fluorinated segments are non-elastomeric polymers with glass transition above room temperature. The hard fluorinated segments may be polymerized from functionalized short chain C₁-C₁₂ alkyl groups. The hard fluorinated segment may be polymerized from fluorinated diamines, fluorinated diisocyanates, or fluorinated diols.

In some embodiments, the hard fluorinated polymer segment includes reactive end groups.

In operation 208, the linkable soft fluoropolymer segment formed in operation 204 is covalently bonded to the hard fluorinated polymer segment having reactive end groups formed in operation 206 to form a hard and soft segmented fluorinated polymer composition. Reactive end groups include, but are not limited to alcohols, amines, thiols or isocyanate groups. When there is more than one hard fluorinated segment, the hard fluorinated segments may be the same hard fluorinated segments, or the composition may include different hard fluorinated segments.

In operation 210, the hard and soft segmented fluorinated polymer composition formed in operation 208 is reacted with a cross-linkable moiety to form hard and soft segmented fluorinated polymer composition with cross-linkable end groups. The cross-linkable moiety may be a methacrylate such as 2-isocyanato ethyl methacrylate, an acrylate such as 2-isocyanato methacrylate, or an epoxide such as glycidol. In some embodiments, the linking moiety and the cross-linkable moiety are the same.

In operation 212, the hard and soft segmented fluorinated polymer composition with cross-linkable end groups formed in operation 210 is crosslinked to form a polymer network. The cross-linking may be accomplished by exposure to ultraviolet (UV) light in the presence of a an alpha hydroxy ketone, phosphine oxide, benzophenone or thioxanthone photoinitiator such as phenylbis(2,4,6-trimethylbenzoyl phosphine oxide or alpha-hydroxycyclohexyl phenyl ketone; or a sulfonium salt, iodonium salt or non-ionic photoacid generator such as triphenyl sulfonium hexafluoro antimonate or triphenylsulfonium hexafluorophosphate. Alternatively, cross-linking may be accomplished thermally, in the presence of one or more thermal initiators. The thermal initiator may be an azo initiator such as azobisisobutyronitrile (AIBN) or 4,4-azobis(4-cyanovaleric acid) (AVCA).

Polymer networks disclosed herein may have several advantageous characteristics which make them suitable for use in electrodes, brain implants, coatings and microelectromechanical systems devices. In some embodiments, the polymer network is transparent to UV and visible light wavelengths.

In some embodiments, the polymer network is an electrical insulator for a range of frequencies of from about 0.1 kHz to about 1 MHZ.

In some embodiments, the polymer network has a number average molecular weight of from about 1,100 to about 1,000,000 g/mol or from about 1,000 to about 20,000 g/mol. In some embodiments, the polymer network exhibits strong adhesion to metal surfaces and thin films.

In some embodiments, the polymer network has a dielectric constant of about 10 or less, about 8 or less, about 6 or less, about 4 or less, about 3 or less, or about 2 or less. In some embodiments, the polymer network has a dielectric constant of from about 1.5 to about 2.5 at 10²-10⁶ Hz.

In some embodiments, the polymer network has a Young's modulus of 150 kPa or less, about 100 kPa or less, about 80 kPa or less, about 60 kPa or less, about 40 kPa or less, about 30 kPa or less, or about 20 kPa or less. In some embodiments, the polymer network has a Young's modulus of from about 1 MPa to about 100 MPa.

In some embodiments, the polymer network is thermally stable at temperatures of up to 300° C., up to about 250° C., up to about 225° C., up to about 200° C. or up to about 175° C.

In some embodiments, the maximum tensile strength of the polymer network is more than 1 MPa, more than 5 MPa, more than 10 MPa, more than 25 MPa, more than 30 MPa, more than 35 MPa, more than 40 mPa or more than 45 MPa.

In some embodiments, the maximum elastic tensile strain of the polymer network is at least about 10%, at least about 20% at least about 30%, at least about 40%, at least about 50%, at least about 75%, at least about 85%, at least about 100%, at least about 125%, at least about 150%, at least about 175%, at least about 200% and up to 225% or greater. In some embodiments, the polymer network exhibits an elastic tensile strain of from about 50% to about 150%.

In some embodiments, the polymer network has an optical refractive index that is similar to that of water or other dilute aqueous solutions, from about 1.2 to about 1.4 or from about 1.29 to 1.39.

In some embodiments, the elasticity of the polymer network is measured as an elongation at break of at least 25%, at least 30%, at least 35%, at least 40%, at least 45%, at least 50%, at least 75%, at least 80%, at least 100%, at least 125%, at least 130%, at least 150%, or at least 175%.

In some embodiments, the polymer network exhibits a combination of advantageous mechanical properties including high tensile strength such as a tensile strength of from about 5 to about 50 MPa; improved toughness; and increased elasticity such as from about 50% to about 150% of elongation at break. In some embodiments, the polymer network has low permeability to water, such as a permeability of from about 10-16 to about 10-18 m²s⁻¹Pa⁻¹.

According to some embodiments, the ultimate true stress of such crosslinked polymer network can be within 1 to 100 MPa and the elongation at break from 100 to 300%. According to some embodiments, the stiffness (Young's modulus) is between 0.1 and 300 MPa.

According to some embodiments, the polymer resin can be used in microfabrication process to form micron scale patterns and to fabricate multi-layer device including polymer and metal layers.

In some embodiment, the copolymer does not absorb light under a range of wavelengths and is transparent. Hence, it is compatible with polymerization under a wide range of wavelengths and does not require photosensitizers.

In some embodiments, the polymer disclosed here, shows improved thermal stability (degradation temperature ˜250° C.) compared to raw polymer (degradation temperature ˜150° C.) while maintaining glass transition temperature well below room temperature.

According to some embodiments, the liquid resin can be cured in either simple or complex molds and can be cut using a die cutter.

According to some embodiments, the dielectric constant is between 1.8 and 3 for a wide range of frequencies i.e. from 100 Hz to 1 MHZ.

For embodiments that involve subsequent exposure of the cured polymer to biological material, such as cells, tissues or organs, the cured polymer may be further purified by washing in polar, semi-polar, non-polar solvents, or in serial washing steps of any selection of these solvents, and then dried. The purified and dried cured polymer may be sterilized by common sterilization modalities, such as ethylene oxide gas, chlorine dioxide gas, or other methods.

The synthetic techniques disclosed can be tuned to produce polymer networks having a specific mechanical property or combination of specific mechanical characteristics described above. Tuning may be achieved by varying the ratios of the various reactants, or by varying the identities of the hard and soft segments, as illustrated in Examples 4-9 below.

While the network properties are tunable, not all combinations of soft and hard segments lead to desirable properties. Maintaining a ratio of hard segments below approximately 30% preserves a spherical domain network morphology, which is necessary for low glass transition, extensibility, and softness. Exceeding 30% hard segments in the polymer backbone results in a higher glass transition and a loss of elastomeric properties, leading to brittleness.

Moreover, an excess of hard segments increases the amount of chain extender required to construct the polymer chain. This leads to increased hydrogen bonding between urethane bonds, resulting in the appearance of hysteresis behavior under tension. Such properties contribute to friction between polymer chains, heat buildup, and non-uniform deformation of the polymer network, which can sometimes be irreversible.

The above-described polymer networks are suitable for use in devices, such as an implantable device including the polymer network and a microelectrode array.

In some embodiments, the device is a neural probe, where at least a portion of a neural probe described herein is implanted in a subject. For example, in various embodiments, part or the entire neural probe may be implanted in a subject. For example, in certain embodiments, the neural probe may be implanted in the brain or other nervous tissue, spinal cord, heart, peripheral muscle, and the like. In some embodiments, the neural probe is configured for long-term residence inside a subject, e.g., the neural probe is stable under physiological conditions. In certain embodiments the neural probe may be configured for long-term contact with the surface of a brain or for partial or full implantation in the brain of subject.

In some embodiments, the polymer network encapsulates the microelectrode array to monitor or stimulate a tissue or an organ electrically. The organ or the tissue includes the brain, central nervous system, spinal cord, skeletal muscle, heart muscle, skin, liver, nasal cavity, spleen, diaphragm, lungs, thyroid, adrenal glands, stomach, eyes, thymus gland, lymph nodes, pancreas, small intestine, ureters, large intestine, bladder, gallbladder, lymphatic vessel, placenta, skeletal muscles, uterus, mouth, prostate, mesentery, pineal gland, subcutaneous tissue, colon, hypothalamus, mammary glands, pituitary gland, cervix, interstitium, parathyroid glands, tonsils, kidneys, or a combination thereof.

The following examples are intended to illustrate certain embodiments of the present invention, but do not exemplify the full scope of the invention. The following examples are offered to illustrate, but not to limit the claimed invention.

### Example 1

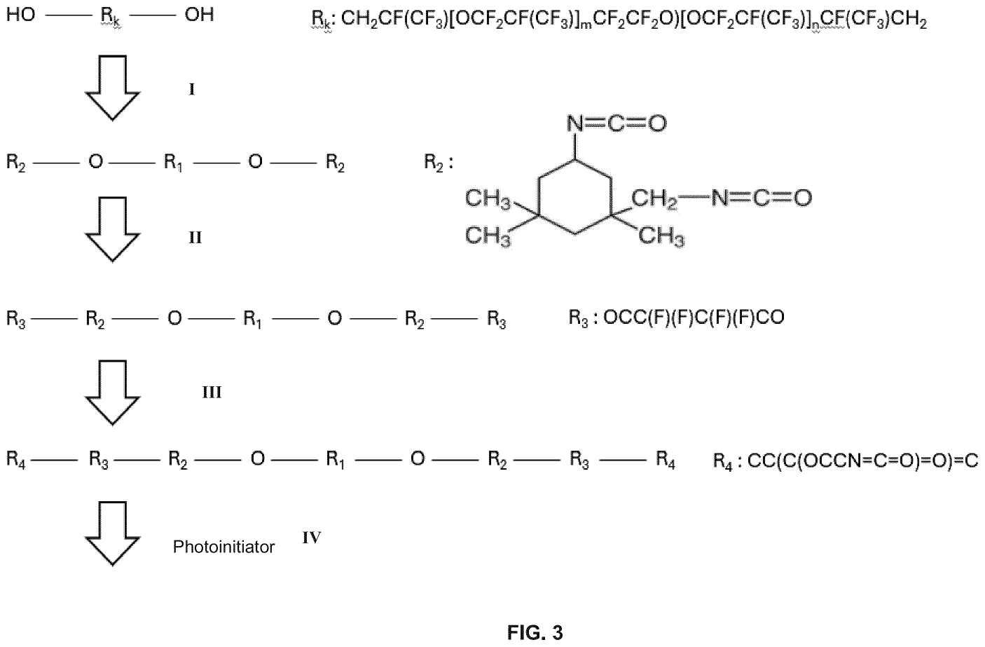

Crosslinked polymer networks can be synthesized as illustrated by the reaction scheme of FIG. 3. In step I of FIG. 3, a soft fluorinated polymer Rx (PFPE including alcohol end groups) was dissolved in 1,3-bis(trifluoromethyl)benzene and heated up to 40° C. in a reaction vessel and then an amount of isophorone diisocyanate (IPDI) was added and the reaction was catalyzed using dibutyltin dilaurate (DBTDL) to form isocyanato linkable end groups. Linkable soft fluoropolymer segments were thus prepared.

In step II of FIG. 3, once all chain-ends were capped with IPDI in step I, the hard fluorinated segments were covalently attached to the linkable soft fluoropolymer segments. To form the hard fluorinated polymer segment, 2,2,3,3-tetrafluoro-1,4-butanediol (TFBD) dissolved in methyl ether ketone was added to the reaction mixture and was allowed to react to completion to form a hard and soft segmented fluorinated polymer composition (also referred to herein as the prepolymer or as a perfluoropolyether copolymer).

In step III of FIG. 3, the prepolymer formed in step II was then reacted with 2-isocyanato ethyl methacrylate (IEM) at room temperature and was catalyzed by existing amounts of DBTDL to form a hard and soft segmented fluorinated polymer composition with cross-linkable end groups. The product was precipitated in a non-solvent to remove catalyst, unreacted isocyanates and impurities.

In step IV of FIG. 3, the crosslinkable functional groups added in step III were crosslinked by photocuring in the presence of a phenylbis(2,4,6-trimethylbenzoyl phosphine oxide (BAPO) initiator.

Crosslinked polymer networks may be synthesized by a process that includes complete solvent extraction or in presence of solvent followed by exposure to ultraviolet (UV) light.

### Example 2

Crosslinked polymer networks were synthesized by dissolving Rₖ in 1,3-bis(trifluoromethyl) benzene and heating up to 40° C. in a reaction vessel. An amount of IPDI was added and the reaction was catalyzed using DBTDL. Once all chain-ends were capped with IPDI, TFBD dissolved in methyl ether ketone was added to the reaction mixture and was allowed to react to completion. The prepolymer was then reacted with IEM at room temperature and was catalyzed by existing amounts of DBTDL to form a reactive polymer. Product was precipitated in a non-solvent to remove catalyst, unreacted isocyanates and impurities. Azobisisobutyronitrile (AIBN) was added as the thermal initiator to form heat curable formulations. Crosslinked polymer networks were synthesized by a process that included complete solvent extraction or in presence of solvent at elevated temperatures. The crosslinked polymer networks created through this process typically exhibit enhanced yield strength and elongation at break, and improved dielectric properties over crosslinked homopolymer networks that are synthesized from similar branched (K-type or Y-type) or linear (Z-type or D-type) without the fluorinated chain extender (hard segments).

### Example 3

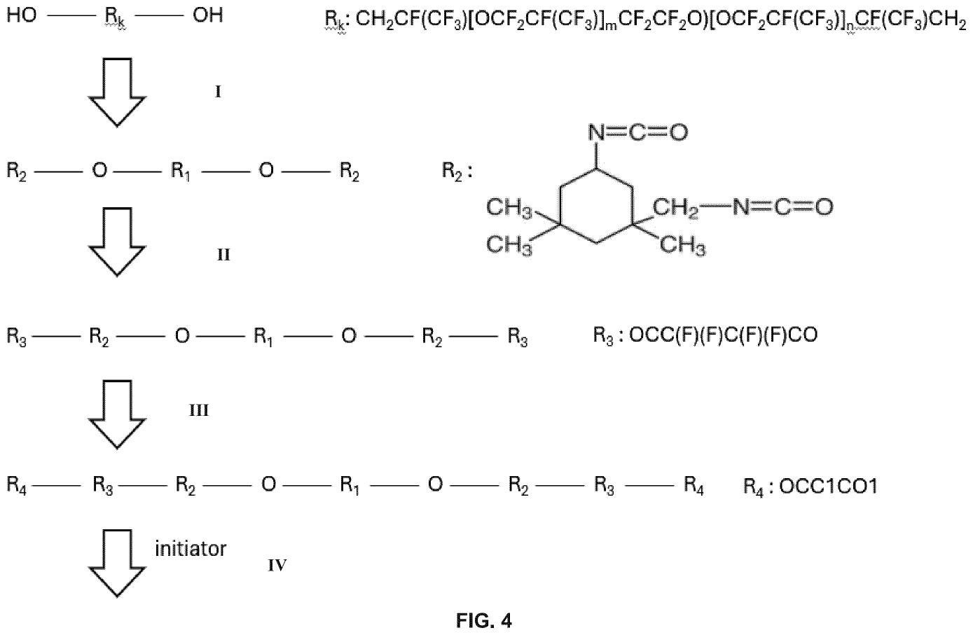

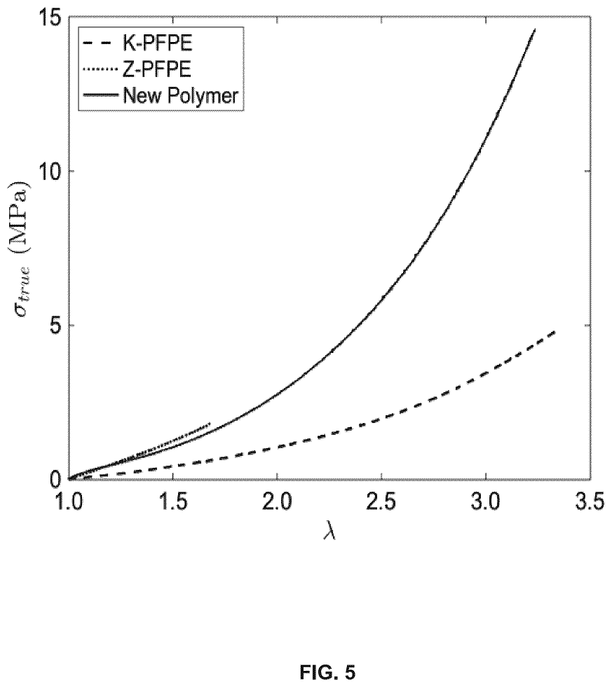

FIG. 4 is a reaction scheme illustrating the synthetic route of Example 3 for forming a polymer network, in accordance with certain disclosed embodiments. In step I of FIG. 4, a soft fluorinated polymer Rₖ including alcohol end groups was dissolved in 1,3-bis(trifluoromethyl) benzene and heated up to 40° C. in a reaction vessel. An amount of IPDI is added and the reaction is catalyzed using DBTDL.

In step II of FIG. 4, once all chain-ends were capped with IPDI in step I, the hard fluorinated segments were covalently attached to the linkable soft fluoropolymer segments. TFBD dissolved in methyl ether ketone was added to the reaction mixture and was allowed to react to completion to form a hard and soft segmented fluorinated polymer composition.

In step III of FIG. 4, the prepolymer formed in step II was then reacted with glycidol at room temperature in the presence of IPDI and was catalyzed by existing amounts of DBTDL to form a hard and soft segmented fluorinated polymer composition with cross-linkable end groups. The product was precipitated in a non-solvent to remove catalyst, unreacted isocyanates and impurities.

In step IV of FIG. 4, the cross-linkable functional groups added in step III were crosslinked in the presence of triphenyl sulfonium hexafluoro antimonate photoacid generator.

Crosslinked polymer networks may be synthesized by a process that includes complete solvent extraction or in presence of solvent followed by exposure to ultraviolet (UV) light.

### Example 4

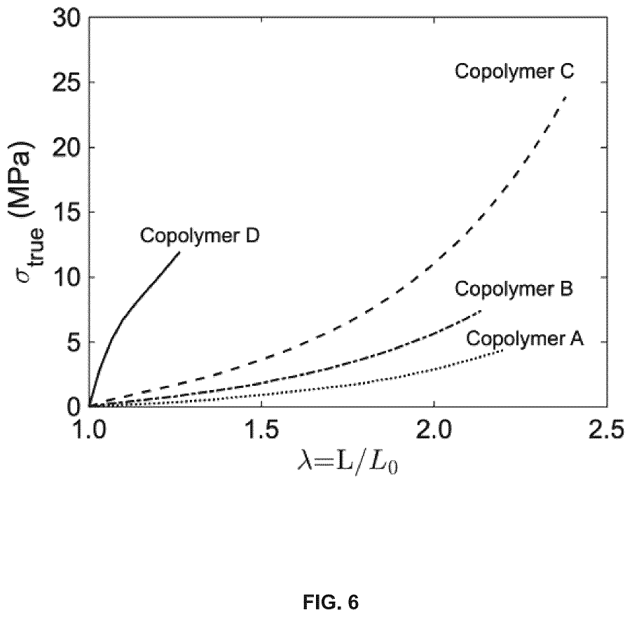

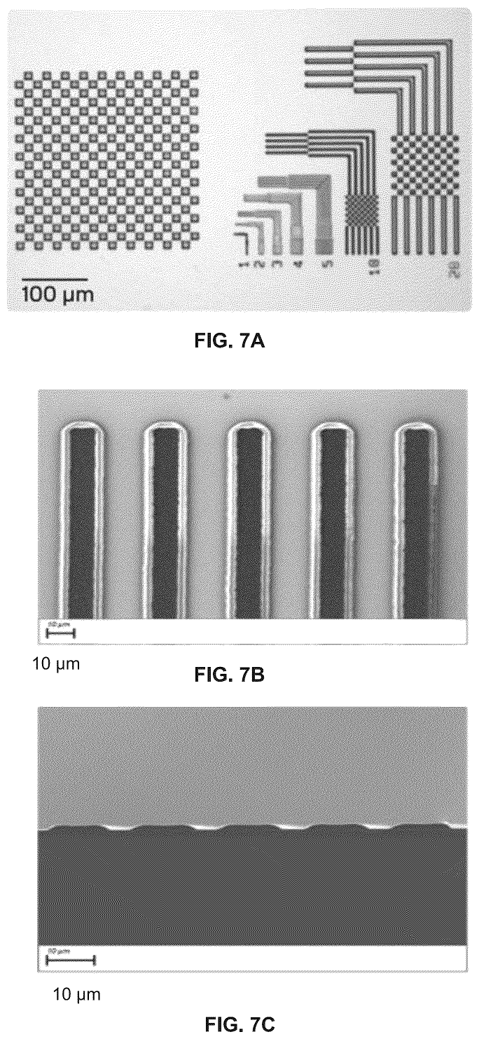

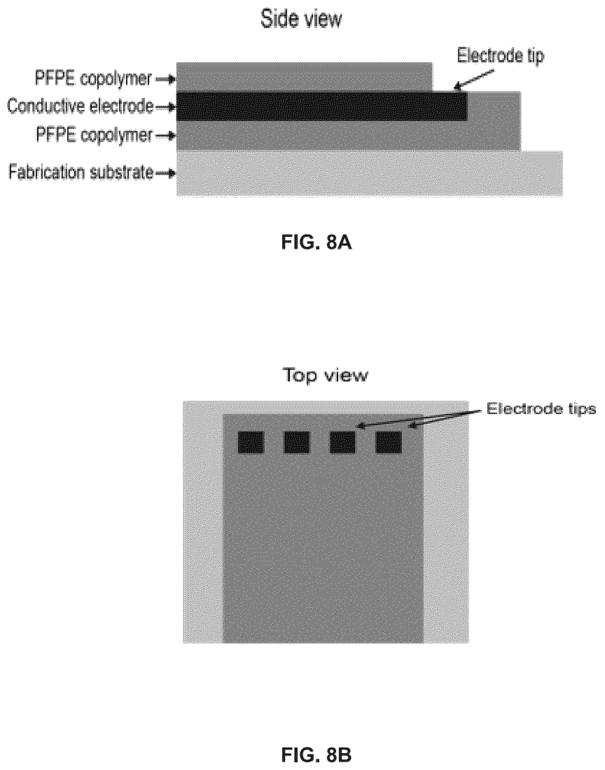

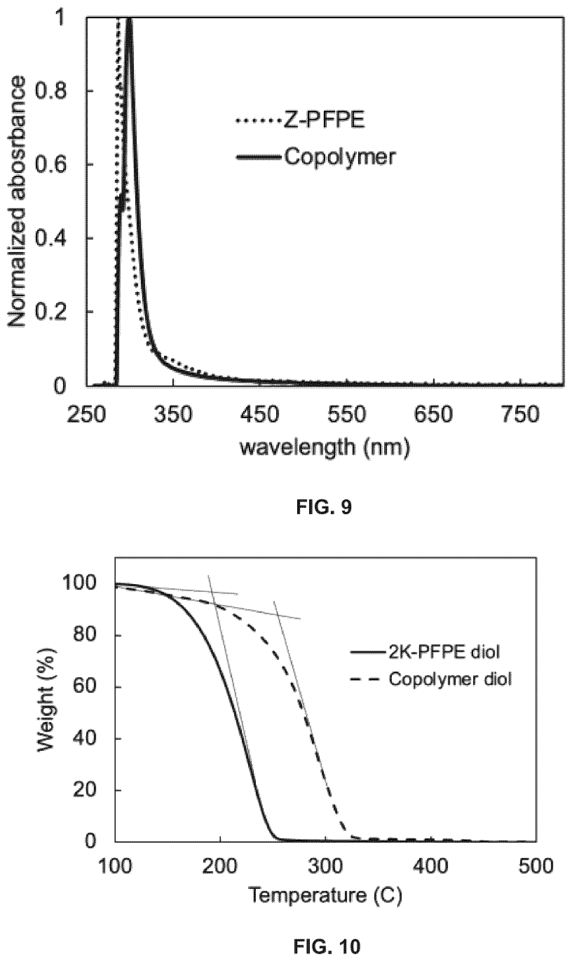

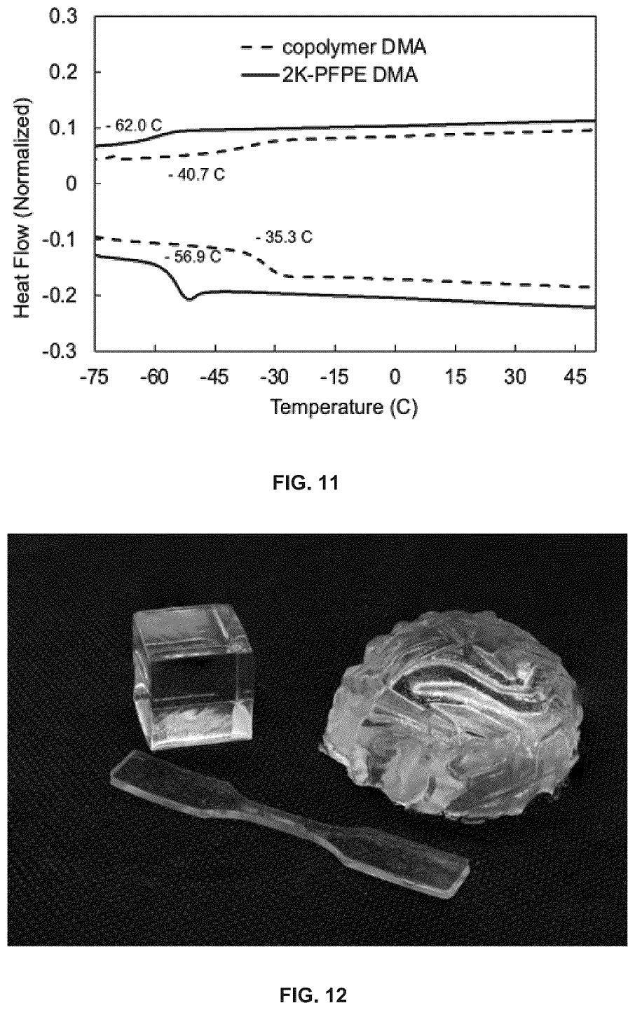

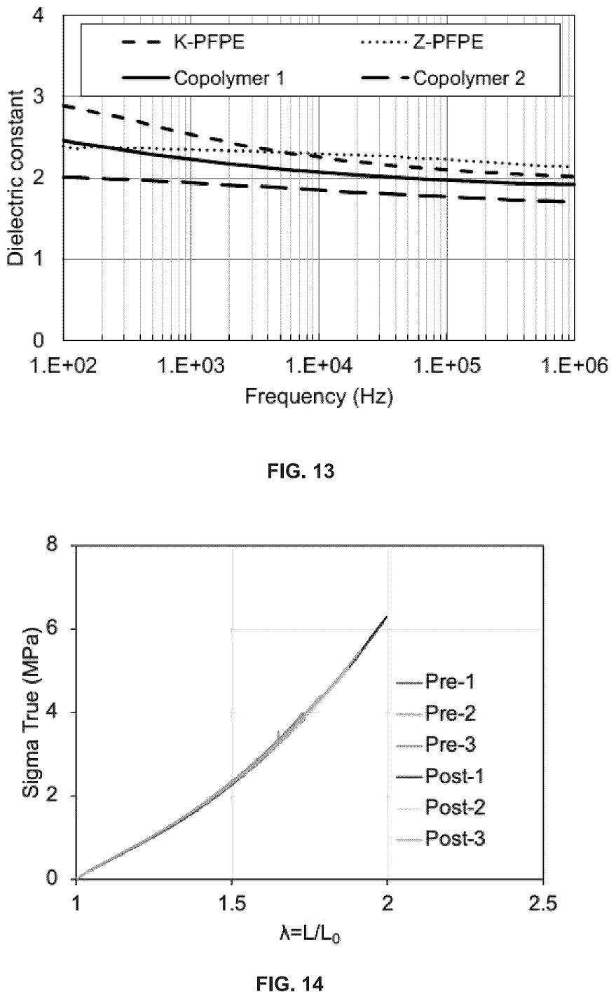

Mechanical properties of crosslinked polymer networks can be tuned by varying the stoichiometric ratio of diisocyanate to PFPE in the synthetic technique of Example 1. Various networks were synthesized by varying the amount of IPDI utilized. The crosslinked polymer networks created through this process showed variation in mechanical properties as shown by the data of FIG. 6.

### Example 5

Mechanical properties of crosslinked polymer networks can also be tuned by varying the stoichiometric ratio of TFBD to PFPE in the synthetic technique of Example 1. Various networks were synthesized by varying the amount of TFBD utilized. The crosslinked polymer networks created through this process showed variation in mechanical properties as shown by the data of FIG. 6.

### Example 6

Mechanical properties of crosslinked polymer networks can alternatively be tuned by varying stoichiometric ratio of two different hard segments at constant PFPE ratio. Such networks were synthesized by dissolution of Rx in 1,3-bis(trifluoromethyl) benzene and heating up to 40° C. in a reaction vessel. An amount of IPDI was added and the reaction was catalyzed using DBTDL. Once all chain-ends were capped with IPDI, different mixture amounts of hard segments including but not limited to TFBD and 1H,1H,11H,11H-perfluoro-3,6,9-trioxaundecane-1,11-diol dissolved in methyl ether ketone in different vials were prepared and added to separate reaction mixtures; and then allowed to react to completion. The prepolymer so formed was then reacted with IEM at room temperature and catalyzed by existing amounts of DBTDL to form a photo reactive polymer. Product was precipitated in a non-solvent to remove catalyst and unreacted isocyanates. BAPO was then added as the initiator to form photocurable formulations. Crosslinked polymer networks were synthesized by a process that includes complete solvent extraction followed by exposure to ultraviolet (UV) light. The crosslinked polymer networks created through this process show variation in mechanical properties as shown by the data of FIG. 6.

### Example 7

Another way to tune the mechanical properties of crosslinked polymer networks is by varying the stoichiometric ratio of multiple different hard segments at constant PFPE ratio in the synthetic technique of Example 1. The crosslinked polymer networks created through this process show variation in mechanical properties.

### Example 8

Mechanical properties of crosslinked polymer networks can additionally be tuned by varying the addition of crosslinkers to PFPE. Various networks were synthesized by dissolving Rₖ in 1,3-bis(trifluoromethyl) benzene and heating up to 40° C. in a reaction vessel. An amount of IPDI was added and the reaction was catalyzed using DBTDL. Once all chain-ends were capped with IPDI, TFBD dissolved in methyl ether ketone and added to the reaction mixture and was allowed to react to completion. The prepolymer was then reacted with IEM at room temperature and catalyzed by existing amounts of DBTDL to form a reactive polymer. The product was precipitated in a non-solvent to remove catalyst and unreacted isocyanates. BAPO and varying amounts of 1,6-hexanedithiol were added as additional crosslinkers/hardeners and the initiator to form photocurable formulations. Crosslinked polymer networks were synthesized by a process that included complete solvent extraction followed by exposure to ultraviolet (UV) light. The crosslinked polymer networks created through this process showed variation in mechanical properties.

### Example 9

Another example of tuning the mechanical properties of crosslinked polymer networks by varying addition of crosslinkers to PFPE is similar to the process described in Example 8, except that BAPO and varying amounts of 1,6-hexanediacrylate was added as additional crosslinker/hardener and the initiator to form photocurable formulations. Crosslinked polymer networks were synthesized by a process that included complete solvent extraction followed by exposure to ultraviolet (UV) light. The crosslinked polymer networks created through this process showed variation in mechanical properties.

### Definitions

As used herein, the term “about” is understood to account for minor increases and/or decreases beyond a recited value, which changes do not significantly impact the desired function of the parameter beyond the recited value(s). In some cases, “about” encompasses+/−10% of any recited value. As used herein, this term modifies any recited value, range of values, or endpoints of one or more ranges.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “elastomeric polymer”, or “elastomer”, refers to any natural or synthetic polymer that is able to resume its original shape when a large deformation is applied. In certain embodiments an elastomeric polymer refers to a polymer or copolymer that, free of diluents, retracts to less than 1.5 times its original length within one minute after being stretched at room temperature (25° C.) to twice its original length and held for one minute before release. Typically a long elastomeric polymer comprises many monomers. The monomers may be covalently bonded. In some cases, the polymer may be modeled as a chain of many links, each link representing a monomer. A large number of polymers can be crosslinked, in some embodiments, by covalent bonds to form a three-dimensional network.

The term “free-standing” refers to the property of an object that does not have the support of a substrate, and can be manipulated and deformed freely within the media it resides in (e.g air, water).

The term “flexible” refers to the physical property of a material being able to be bent without breaking at certain dimensions, it does not imply about the Young's modulus of the material and stretchability.

The term “segment” refers to a short chain of functional polymers.

The term “hard” refers to the physical property of a material that resists mechanical deformation.

The term “soft” refers to the physical property of a material that deforms or yields readily to pressure or weight; and easily deforms upon mechanical stress. These materials usually have a Young's modulus of less than 1 GPa and can be stretched up to 5% without breaking.

The term “polymer network” refers to a three-dimensional structure composed of polymer chains conformed by chemical interconnection or cross-linking.

The term “device” refers to articles such as sensors, implants, circuits, or coated substrates.

The term “probe” refers to a structure comprising one or a plurality of electrodes configured delivering a signal to or receiving a signal from a biological tissue, e.g., the brain or other neurological tissue, the heart or other organs.

The term “neural probe” refers to a structure comprising one or a plurality of electrodes configured delivering a signal to or receiving a signal from the brain or other neurological tissue such as in the peripheral nervous system or the heart. In various embodiments the neural probe comprises one or a plurality of flexible electrodes, e.g., microelectrodes, deposited on or in one or more flexible polymer layer(s).

An “electrode” refers to a conductive element configured to conduct charge from a first point to a second point. In various embodiments an electrode can comprise one or more “tip(s)” or “contact area(s)”, a conductor region, and a terminal region. In certain embodiments the tip(s) are configured for contact with a tissue, e.g., brain or other neural tissue, while the contact areas are configured to facilitate electrical connection to one or more electrical components.

The term “flexible polymer”, or “polymer”, refers to any polymeric material that can be bent without breaking down and can resume to its original shape after the deformation. This flexible polymer material can be an inherently elastomer, namely a natural or synthetic polymer that is able to resume its original shape when a large deformation is applied. In certain embodiments an elastomeric polymer refers to a polymer or copolymer that, free of diluents, retracts to less than 1.5 times its original length within one minute after being stretched at room temperature to twice its original length and held for one minute before release. This flexible polymer can also be a plastic material or resin material, that in the bulk form factor is hard and brittle, but then made into thin films with a thickness less than 10 μm, it can be bent without breaking.

A “fluoropolymer” is a fluorocarbon-based polymer with multiple carbon-fluorine bonds. Illustrative fluoropolymers include, but are not limited to PVF (polyvinylfluoride), PVDF (polyvinylidene fluoride), PTFE (polytetrafluoroethylene), PCTFE (polychlorotrifluoroethylene), PFA, MFA (perfluoroalkoxy polymer), FEP (fluorinated ethylene-propylene), ETFE (polyethylenetetrafluoroethylene), ECTFE (polyethylenechlorotrifluoroethylene), FFPM/FFKM (Perfluorinated Elastomer [Perfluoroelastomer]), FPM/FKM (Fluoroelastomer [Vinylidene Fluoride based copolymers]), FEPM (Fluoroelastomer [Tetrafluoroethylene-Propylene]), PFPE (Perfluoropolyether), PFSA (Perfluorosulfonic acid), and the like.

A perfluoropolymer is a polymer derived from another by replacing all (or most) of the hydrogen atoms by those of fluorine. Typically, a perfluoropolymer is a polymer wherein the carbon atoms within all or a portion of the polymer are only bound to fluorine and/or other heteroatoms, rather than hydrogen. A perflurorelastomer is an elastomer where the carbon atoms within all or a portion of the elastomer are only bound to fluorine and/or other heteroatoms, rather than hydrogen.

The term “physiological conditions” as used herein, refers to conditions typical inside the body of a mammal, e.g., conditions simulating those under which the (normal) functions of a cell, organ, or tissue can be expressed. Illustrative physiological conditions can comprise approximately neutral pH (e.g., pH 7.0-7.4), salinity of about 9-10% (e.g., about 0.1 to about 0.2 M NaCl or about 0.15 M NaCl), temperature ranging from about 96° F. to 104° F. (˜35° C. to ˜40° C.) and the like. A typical temperature, for humans is about 37° C.

The term “stably bonded” when referring to a multi-layered article e.g., as described herein, indicates that the layers typically do not delaminate under physiological conditions, e.g., when implanted into a tissue or organ of a mammal. Typically, when the layers are stably bonded, they remain bonded under physiological conditions for at least 1 week, or at least 2 weeks, or at least 3 weeks, or at least 1 month, or at least 2 months, or at least 3 months, or at least 4 months, or at least 5 months, or at least 6 months, or at least 7 months, or at least 8 months, or at least 9 months, or at least 10 months, or at least 11 months, or at least 1 year, or at least 1.5 years, or for at least 2 years.

The terms “subject,” “individual,” and “patient” may be used interchangeably and refer to humans, as well as non-human mammals (e.g., non-human primates, canines, equines, felines, porcines, ovines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

A “capacitive electrode” is an insulated electrode that does not make ohmic contact with tissues or body fluids.

The term “integrated circuit” refers to group of electronic circuits or devices and their connections that are small and are produced in or on a small slice of material (e.g., silicon) or in whole a wafer format.

A “circuit element” or “integrated circuit element” refers to a component of an integrated circuit. The element can be a device comprising the integrated circuit, including but not limited to, a preamplifier, a multiplexer, a voltage regulator, analog to digital converter (ADC), digital to analog converter (DAC), microcontroller, field programmable gate array (FPGA), transceiver, signal conditioner, or memory device, or a connection/interconnect to a device comprising the integrated circuit.

### CONCLUSION

Although the foregoing embodiments have been described in some detail for purposes of clarity of understanding, it will be apparent that certain changes and modifications may be practiced within the scope of the appended claims. It should be noted that there are many alternative ways of implementing the processes, systems, and apparatus of the present embodiments. Accordingly, the present embodiments are to be considered as illustrative and not restrictive, and the embodiments are not to be limited to the details given herein.

What is claimed is:

1. A composition comprising:
at least one soft fluoropolymer segment; and
at least one hard fluorinated polymer segment; and
wherein the at least one soft fluoropolymer segment and the at least one hard fluorinated polymer segment are covalently bound through a linking moiety comprising at least two reactive groups.

2. The composition of claim 1, wherein the at least one soft fluoropolymer segment comprises poly(1,1,1,3,3,3,-hexafluoroisopropyl acrylate) (PHFIPA), poly[2-(perfluorohexyl)ethyl]acrylate, perfluoropolyether (PFPE), polytetrafluoroethylene (PTFE), tetrafluoroethylene propylene (TFE), perfluoropolyether dimethyl acrylate (PFPE-DMA), fluorinated ethylene-propylene (FEP), perfluoroalkoxy polymer (PFA), or polychlorotrifluoroethylene (PCTFE).

3. The composition of claim 2, wherein the at least one soft fluoropolymer segment comprises a perfluoropolyether having a number average molecular weight Mn of from about 1,000 to about 10,000 g/mol.

4. The composition of claim 3, wherein the perfluoropolyether further comprises at least one cross-linkable moiety.

5. The composition of claim 4, wherein the cross-linkable moiety comprises a methacrylate, an acrylate, or an epoxide.

6. The composition of claim 4, wherein the linking moiety and the cross-linkable moiety are the same moiety.

7. The composition of claim 4, wherein the linking moiety comprises a multi-functional isocyanate.

8. The composition of claim 7, wherein the multi-functional isocyanate comprises isophorone diisocyanate (IPDI), hexamethylene diisocyanate (HDI), 4,4′-diisocyanato dicyclohexylmethane (HMDI), methylene diphenyl diisocyanate (MDI), 2,2′-MDI, 2,4′-MDI, 4,4′-MDI, toluene diisocyanate (TDI), 1,3,5-tris(6-isocyanatohexyl)-1,3,5-triazinane-2,4,6-trione, 1,3,5-tris[(5-isocyanato-1,3,3-trimethylcyclohexyl)methyl]-1,3,5-triazinane-2,4,6-trione, 1,3,5-triazine-2,4,6 (1H,3H,5H)-trione or a combination thereof.

9. The composition of claim 3, wherein the hard fluorinated polymer segment is formed of a fluorinated diol; and the fluorinated diol comprises hexafluoro-2,3-bis(trifluoromethyl)-2,3-butanediol, 2,2,3,3-tetrafluoro-1,4-butanediol (TFBD), 2,2,3,3,4,4-hexafluoro-1,5-pentanediol, 2,2,3,3,4,4,5,5-octafluoro-1,6-hexanediol, 2,2,3,3,4,4,5,5,6,6,7,7,8,8,9,9-hexadecafluoro-1,10-decanediol, 1H,1H,10H,10H-perfluoro-1,10-decanediol, or a combination thereof.

10. The composition of claim 8, wherein the linking moiety comprises isophorone diisocyanate.

11. A polymer network comprising:
soft fluoropolymer segments; and
hard fluorinated polymer segments; and
wherein the soft fluoropolymer segments and the hard fluorinated polymer segments are covalently bound through a linking moiety comprising at least two reactive groups;
and wherein the soft fluoropolymer segments are connected through cross-linkers.

12. The polymer network of claim 11, wherein the polymer network has a dielectric constant of from about 1 to about 5.

13. The polymer network of claim 11, wherein the polymer network has a Young's modulus of from about 1 MPa to about 100 MPa.

14. The polymer network of claim 11, wherein the polymer network has a dielectric constant of from about 1.5 to about 3 and a Young's modulus of from about 25 MPa to about 50 MPa.

15. The polymer network of claim 11, wherein the polymer network is transparent to UV and visible light wavelengths.

16. The polymer network of claim 11, wherein the polymer network has an optical refractive index of from about 1.2 to 1.4.

17. The polymer network of claim 11, wherein the polymer network is an electrical insulator for a range of frequencies of from about 0.1 kHz to about 1 MHz.

18. The polymer network of claim 11, wherein the polymer network has low permeability to water.

19. The polymer network of claim 11, wherein the polymer network has a number average molecular weight of from about 1,100 to about 1,000,000 g/mol.

20. The polymer network of claim 11, wherein the polymer network exhibits mechanical properties of high tensile strength, improved toughness, and increased elasticity.

21. The polymer network of claim 11, wherein the polymer network has an elongation at break of from about 50% to about 150%.

22. The polymer network of claim 11, wherein the polymer network exhibits strong adhesion to metal surfaces and thin films.

23. The polymer network of claim 11, wherein the polymer network is stable at a temperature of up to 300° C.

24. The polymer network of claim 11, for use in electrodes, brain implants, coatings, or microelectromechanical systems (MEMS) devices.

25. A device comprising:
the polymer network of claim 11,
and a microelectrode array.

26. The device of claim 25, wherein the polymer network encapsulates the microelectrode array to monitor or stimulate a tissue or an organ electrically.

27. The device of claim 26, wherein the organ or the tissue comprises brain, central nervous system, spinal cord, skeletal muscle, heart muscle, skin, liver, nasal cavity, spleen, diaphragm, lungs, thyroid, adrenal glands, stomach, eyes, thymus gland, lymph nodes, pancreas, small intestine, ureters, large intestine, bladder, gallbladder, lymphatic vessel, placenta, skeletal muscles, uterus, mouth, prostate, mesentery, pineal gland, subcutaneous tissue, colon, hypothalamus, mammary glands, pituitary gland, cervix, interstitium, parathyroid glands, tonsils, kidneys, or a combination thereof.

28. A method of making a polymer network comprising:
providing at least one soft fluoropolymer comprising reactive end groups;
attaching a linking moiety through the reactive end groups of the at least one soft fluoropolymer to form a linkable soft fluoropolymer segment;
polymerizing a fluorinated moiety to form a hard fluorinated polymer segment comprising reactive end groups;
covalently bonding the linkable soft fluoropolymer segment to the hard fluorinated polymer segment comprising reactive end groups to form a hard- and soft-segmented fluorinated polymer composition;
reacting the hard- and soft-segmented fluorinated polymer composition with a cross-linkable moiety to form a hard- and soft-segmented fluorinated polymer composition with cross-linkable end groups; and
cross-linking the hard- and soft-segmented fluorinated polymer composition with cross-linkable end groups to form the polymer network.

29. The method of claim 28, wherein the at least one soft fluoropolymer comprises poly(1,1,1,3,3,3,-hexafluoroisopropyl acrylate) (PHFIPA), poly[2-(perfluorohexyl)ethyl]acrylate, perfluoropolyether (PFPE), polytetrafluoroethylene (PTFE), tetrafluoroethylene propylene (TFE), perfluoropolyether dimethyl acrylate (PFPE-DMA), fluorinated ethylene-propylene (FEP), perfluoroalkoxy polymer (PFA), or polychlorotrifluoroethylene (PCTFE).

30. The method of claim 29, wherein the at least one soft fluoropolymer comprises a K-type, a D-type, a Y-type, or a Z-type perfluoropolyether having a number average molecular weight Mn of from about 1,000 to about 10,000 g/mol.

31. The method of claim 28, wherein the cross-linkable moiety comprises an acrylation or methacrylation reagent; and wherein the hard- and soft-segmented fluorinated polymer composition with cross-linkable end groups is cross-linked into a polymer network by free radical polymerization in the presence of a thermal initiator, a photoinitiator, or a combination thereof.

32. The method of claim 28, wherein the cross-linkable moiety comprises an epoxy reagent; and wherein the hard- and soft-segmented fluorinated polymer composition with cross-linkable end groups is cross-linked into a polymer network using a photoacid generator.

33. The method of claim 28, wherein the fluorinated moiety comprises a fluorinated diamine, a fluorinated diisocyanate, a fluorinated diol or a combination thereof.

34. The method of claim 28, wherein each of the fluorinated moiety and the linking moiety are the same.

35. The method of claim 33, wherein each of the fluorinated moiety and the linking moiety are the same fluorinated or perfluorinated diisocyanate.

36. The method of claim 28, wherein covalent bonding of the linkable soft fluoropolymer segment to at least one hard fluorinated polymer segment is catalyzed by dibutyltin dilaurate (DBTDL).

37. The method of claim 28, wherein increasing the number of the hard fluorinated polymer segments in the polymer network controls the mechanical properties of the polymer network.

38. The method of claim 28, further comprising sterilization of the polymer network by gamma irradiation, e-beam irradiation, ethylene oxide, chlorine dioxide, nitrogen dioxide, hydrogen peroxide, UV irradiation, dry heat, steam or a combination thereof.

39. The method of claim 28, further comprising patterning the polymer network on a substrate with a developing solvent, to provide a pattern with a lateral and vertical resolution below 100 μm.

40. The method of claim 28, further comprising processing the polymer network by injection molding, spin coating, dip coating, solvent casting, extrusion, electrospinning, thermal drawing, hot embossing, inkjet printing, stereolithography, fused deposition molding, imprinting or a combination thereof.

41. The method of claim 28, further comprising purifying the polymer network by precipitation.
